(19) United States
(12) Patent Application Publication
(10) Pub. No.: US 2013/0165949 A1
(43) Pub. Date: Jun. 27, 2013

(54) SOFT TISSUE COAPTOR AND DEVICE FOR DEPLOYING SAME

(71) Applicants: Jeffrey Olson, Cherry Hills Village, CO (US); Bryan Rech, Boulder, CO (US); Craig Lanning, Denver, CO (US); Mike Erlanger, Denver, CO (US); Robin Shandas, Boulder, CO (US)

(72) Inventors: Jeffrey Olson, Cherry Hills Village, CO (US); Bryan Rech, Boulder, CO (US); Craig Lanning, Denver, CO (US); Mike Erlanger, Denver, CO (US); Robin Shandas, Boulder, CO (US)

(73) Assignee: The Regents of the University of Colorado , a Body Corporate, Denver, CO (US)

(21) Appl. No.: 13/635,565

(22) PCT Filed: Mar. 17, 2011
(86) PCT No.: PCT/US2011/028877

§ 371 Date: Dec. 14, 2012

Related U.S. Application Data
(60) Provisional application No. 61/315,718, filed on Mar. 19, 2010.

(51) Int. Cl. A61B 17/10

(52) U.S. Cl. CPC A61B 17/10
USPC 606/139

(57) ABSTRACT

A soft tissue coaptor and a soft tissue coaptor deployment device are disclosed which deliver a soft tissue coaptor or clip to a desired location for tissue repair. The soft tissue coaptor and soft tissue coaptor deployment device have particular application in surgical and microsurgical settings such as cardiac, vascular, and ophthalmic surgery.

### FIELD OF INVENTION

The present invention generally relates to a biocompatible soft tissue coaptor made from composite shape memory materials and a deployment device for accurately positioning and securing the same for use in surgical and microsurgical settings. More particularly, the present invention relates to a minimally invasive device which is capable of easily and accurately delivering a biocompatible soft tissue coaptor during small scale minimally invasive surgery. For example, the device of the present invention is particularly useful in ophthalmic surgeries such as coaption of iris defects, intraocular implants, glaucoma tube shunts, or other implantable hardware for the eye. The device is also particularly useful in other applications such as cardiac surgery, vascular surgery, and any other small scale minimally invasive surgeries.

### BACKGROUND OF THE INVENTION

Many types of suturing and securing devices and procedures exist for surgical and microsurgical settings. In particular, with respect to ophthalmic surgeries, laser treatments, micro scissors, and implants having nylon clip members may be used to access and manipulate soft tissue for correction of various conditions and defects.

In order to enable more efficient and accurate correction of such conditions and defects, there is a need for a securing device or mechanism and a deployment device for the same that can be used in minimally invasive surgery settings, especially ophthalmic surgery settings.

### SUMMARY OF THE INVENTION

The present invention is directed to a soft tissue coaptor and a soft tissue coaptor deployment device which delivers a soft tissue coaptor to a location within the body during minimally invasive tissue repair, implantation surgeries and the like. The device can be used in many applications including, but not limited to, cardiac surgery, vascular surgery, ophthalmic surgery, and other applications that require minimally invasive surgery and/or tissue repair.

The device is particularly useful in ophthalmic surgeries and procedures including coaptation in iris defects, securing or fixing in place intraocular implants such as intraocular lenses, glaucoma tube shunts, implantable hardware for the eye, securing full and partial thickness cornea transplants, LASIK and DSEK/DLEK flaps, temporary and permanent keratoprostheses, implantable ocular lenses, contact lenses and telescopic lenses, presbyopia reversal, scleral patch grafts and scleral rings, conjunctival and amniotic membrane grafts, repair of the iris and isis root defects, iridoplasty, pupiloplasty, securing dislocated intraocular lenses to the iris, anchoring the capsular bag, anchoring capsular tension rings, corneal wound closure, anchoring tube shunts both in the anterior chamber and externally to the sclera, closure of sclerotomies, conjunctival flaps, trabeculotomy and trabeculectomy blebs, closure of cyclodialysis clefts, fixation of intraocular pressure monitoring devices, fixation of intraocular implants for sustained drug delivery, anchoring orbital reconstruction hardware, weighted lid implants, eyelid skin and muscle wound closure, fixation of lacrimal system hardware, tarsorraphy, repair of ptosis, blepharoplasty, correction of entropion and ectropion, canthoplasty, fixation of virectomy infusion line, closure of sclerotomies, scleral buckling with or without silicone band or sponge hardware, retinopexy, closure of traumatic corneal and scleral wounds, fixation of radioactive plaques for the treatment of intraocular tumors, fixation of intraocular hardware and implantable chips for artificial vision and electrical stimulation of the retina, correction of blepharospasm, and fixation of extraocular muscles to sclera for resection, recession, and transposition surgeries.

The soft tissue coaptor of the present invention is biocompatible and is, in exemplary embodiments, made of a shape memory material. An exemplary shape memory material may posses the property of having at least two distinctive configurations and the ability to transform between these configurations given the application or removal of an external stimulus such as strain, heat and/or light. In a preferred embodiment, an exemplary shape memory material is capable of being deformed without inducing any permanent deformation.

In one exemplary embodiment, the soft tissue coaptor of the present invention is comprised of a shape memory material and has a sharpened proximal end for piercing tissue. In a first configuration, before deployment, the coaptor is long and straight in order to optimize delivery into the body by minimizing the coaptor's cross sectional profile. After a specified time period, determined by the material properties, or, in the alternative, after the application or removal of an external stimulus, the coaptor will undergo a transformation to a second configuration optimized for fixation of implants and tissues in the eye. In one exemplary embodiment, the first configuration is a straight line configuration that is optimized for minimizing the incision, the second configuration is a circular configuration that is optimized for coaption, and the stimulus for the transformation from the first configuration to the second configuration is body heat. Another example of this embodiment may include clips or coapters that have curved features that follow the geometry of the eye. These clips or coaptors would be custom made based on radiological images of the patient. Unlike conventional surgical stapling systems, which pinch tissue between the tines of a staple applied to the surface, the injectable clip system described below may be configured in an exemplary embodiment, to actually penetrate tissue, and be deployed beneath the surface.

The deployment device of the present invention for delivering and securing a soft tissue coaptor at a surgical site includes a hollow housing having a. first end and a second end, a hollow deployment chamber or body positioned within, and/or extending from, the first end of the hollow housing, and an actuator mechanism positioned within, and/or extending from, the second end of the hollow housing. In one exemplary embodiment, the hollow housing and the hollow deployment chamber may both be cylindrical in shape or have a “tube” shape. One or more coaptors are loaded into the hollow deployment chamber before utilizing the deployment device to deliver and secure the coaptor. In one exemplary embodiment, the hollow deployment chamber is configured to anchor the coaptor or clip within the deployment device, for example, by frictional engagement.

In one exemplary embodiment, the actuator mechanism is configured to anchor a distal end of the coaptor or clip within the deployment device, for example, by frictional engagement, mechanical engagement, adhesion, etc. In this manner, the actuator mechanism may be configured to deploy the coaptor from the deployment device once the coaptor is in a desired position. Similarly, the actuator mechanism may be configured to retract the coaptor so that it may be repositioned if necessary.

In another exemplary embodiment, the actuator mechanism includes an actuator rod contained within the second end of the hollow housing and an ejector pin contained within, and/or extending from, the actuator rod that is capable of fitting within the hollow deployment chamber. The proximal end of the ejector pin may be completely contained within the hollow deployment chamber at all times and the hollow deployment chamber may have a sharpened proximal end for piercing tissue to allow access to a deployment site.

In another exemplary embodiment, the deployment device of the present invention may also include one or more finger supports attached to an outer surface of the hollow housing for supporting a user's fingers while employing the actuator mechanism of the deployment device in order to allow for greater control while utilizing the deployment device. The finger support may include a single opening or two openings on opposite sides of the hollow housing for inserting a user's finger(s) therethrough.

In yet another exemplary embodiment, the deployment device of the present invention may include a thumb support attached to the actuator mechanism for supporting a user's thumb while employing the actuator mechanism. The thumb support may comprise a post or include an opening for inserting a user's thumb therethrough.

In still another exemplary embodiment, the actuator mechanism may include a geared or ratcheted system that deploys the coapter by either rotation of the deployment chamber or compression of a button or lever or a system that deploys the coapter by twisting, wheeled action or sliding. Alternatively, in yet another exemplary embodiment, the actuator mechanism may comprise a pneumatically driven system or a hydraulically driven system such as those currently known in the art. In yet another exemplary embodiment, the actuator mechanism of the present invention may be capable of interfacing with a foot pedal or a trigger device configured to control deployment of the coaptor.

In another exemplary embodiment of the deployment device, the deployment device may include a mechanism capable of retracting the coaptor from the body. Alternatively, an external stimulus such as, for example, strain, heat and/or light may be applied to or removed from the coaptor to transform the coaptor back to its first configuration, i.e. its configuration before deployment. In yet another exemplary embodiment, the actuator mechanism of the present invention can be a viscoelastic that deploys the coaptor.

In yet another exemplary embodiment, the deployment device of the present invention may be capable of interfacing with a robotic arm for remote or telescopic surgery.

### BRIEF DESCRIPTION OF THE DRAWINGS

A more complete understanding of the present invention may be derived by referring to the detailed description and claims when considered in connection with the following illustrative Figures, which may not be to scale. In the following Figures, like reference numbers refer to similar elements throughout the Figures.

The drawings serve to illustrate embodiments of the invention and the invention is not limited to these, but certain of the drawings schematically present exemplary embodiments of the invention, as disclosed herein.

FIGS. 1A and 1B each comprise a perspective view of an exemplary embodiment of the soft tissue coaptor of the present invention shown in a second configuration, which is the configuration it is in after implantation (i.e. the implantation configuration).

FIG. 2 is a perspective view of the soft tissue coaptor of FIG. 1A shown in a first configuration, which is the configuration it is in when loaded within the deployment device of the present invention.

FIG. 3 is a perspective view of an exemplary embodiment of the deployment device of the present invention.

FIG. 4 is a cross-sectional view taken along line 5-5 of FIG. 3.

FIGS. 5A-5C are views of a coapter being deployed from the sharpened proximal end of the deployment device shown in FIG. 3.

FIG. 6 is a top plan view of the deployment device shown in FIG. 3.

FIGS. 7A and 7B each comprise a view of an exemplary embodiment of the deployment device of the present invention.

FIGS. 8A and 8B are column graphs illustrating improvements over the prior art in corneal wound applications.

FIGS. 9A and 9B are column graphs illustrating improvements over the prior art in iris repair and intraoclular lens (“IOL”) fixation applications respectively.

### DETAILED DESCRIPTION

Persons skilled in the art will readily appreciate that various aspects of the present invention may be realized by any number of methods and apparatuses configured to perform the intended functions. Stated differently, other methods and apparatuses may be incorporated herein to perform the intended functions. Finally, although the present invention may be described in connection with various principles and beliefs, the present invention should not be hound by theory.

The present invention includes a biocompatible soft tissue coaptor made of a shape memory material and a deployment device for delivering and securing the soft tissue coaptor to a desired position during surgery. The present invention has particular application in cardiac, vascular, and ophthalmic surgery and more widespread applications are also contemplated, including various minimally invasive type surgeries such as laproscopic/endoscopic general surgery including, orthopedic, cardiac, general surgery, obstetric/gynecology, urology, interventional radiology, neurosurgical, vascular etc.

With respect to ophthalmic applications, the soft tissue coaptor and deployment device of the present invention are particularly useful for coaptation in iris defects, securing or fixing in place intraocular implants such as intraocular lenses, glaucoma tube shunts, implantable hardware for the eye, securing full and partial thickness cornea transplants, LASIK and DSEK/DLEK flaps, temporary and permanent keratoprostheses, implantable ocular lenses, contact lenses and telescopic lenses, presbyopia reversal, scleral patch grafts and scleral rings, conjunctival and amniotic membrane grafts, repair of the iris and iris root defects, iridoplasty, pupiloplasty, securing dislocated intraocular lenses to the iris, capsular bag fixation, anchoring capsular tension rings, corneal wound closure, anchoring tube shunts both in the anterior chamber and externally to the sclera, closure of sclerotomies, conjunctival flaps, trabeculotomy blebs, closure of cyclodialysis clefts, fixation of intraocular pressure monitoring devices, fixation of intraocular implants for sustained drug delivery, anchoring orbital reconstruction hardware, weighted lid implants, eyelid skin and muscle wound closure, fixation of lacrimal system hardware, tarsorraphy, repair of ptosis, blepharoplasty, correction of entropion and ectropion, canthoplasty, fixation of virectomy infusion line, closure of sclerotomies, scleral buckling with or without silicone band or sponge hardware, retinoplexy, closure of traumatic corneal and scleral wounds, fixation of radioactive plaques for the treatment of intraocular tumors, fixation of intraocular hardware and implantable chips for artificial vision and electrical stimulation of the retina, correction of blepharospasm, and fixation of extraocular muscles to sclera for resection, recession, and transposition surgeries.

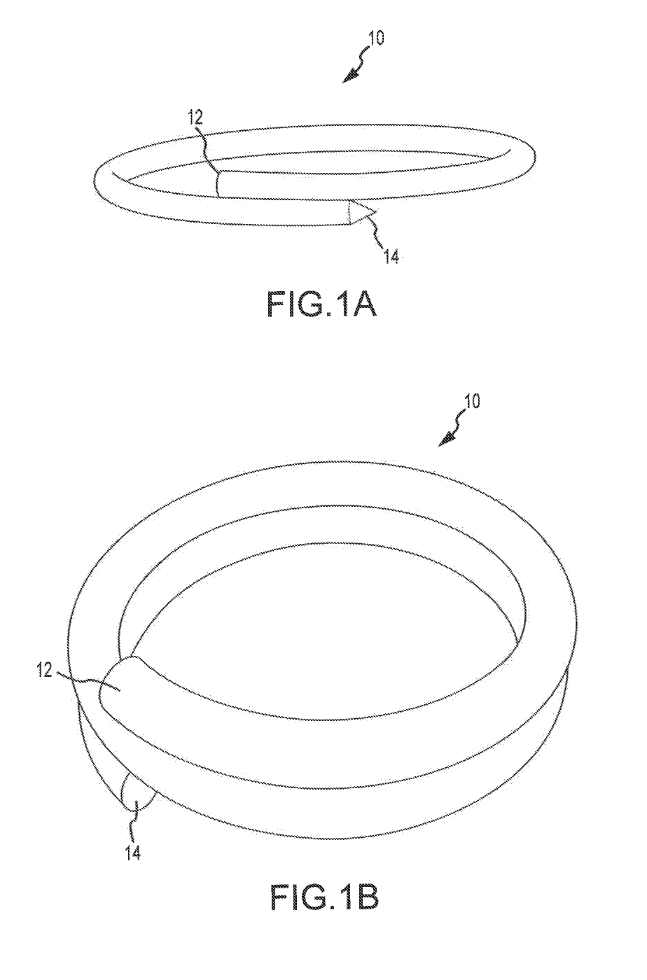

A perspective view of an exemplary embodiment of the soft tissue coaptor of the present invention shown in its implanted, or secondary, configuration is shown in FIGS. 1A and 1B. Soft tissue coaptor 10 has a distal end 12 and a proximal end 14. “Proximal,” as used herein, refers to the end that is closer to the surgical site during deployment of the coaptor, while “distal” refers to the end that is closer to the user of the deployment device. With reference to FIG. 1A, in another exemplary embodiment, proximal end 14 may be sharpened for piercing tissue upon its deployment from the deployment device of the present invention. With reference to FIG. 1A, proximal end 14 may be blunt, for example, for blunt dissection upon its deployment from the deployment device. In yet other exemplary embodiments, proximal end 14 may be configured to have one or more of a barb, corkscrew, hook, grappling hook and hollow point.

In exemplary embodiments, soft tissue coaptor 10 is comprised of a biocompatible shape memory material such as nitinol so that it is capable of being deformed while being utilized with the deployment device without inducing any permanent deformation. In exemplary embodiments, soft tissue coaptor 10 is comprised of a MRI safe material such as nitinol.

In exemplary embodiments, soft tissue coaptor 10 is surface treated to increase biocompatibility. Surface modification to improve biocompatibility may comprise one or more of chemical, laser, plasma, ion implantation, chemical vapor deposits, bioactive surface (plasma/fibronectin), electrochemical processing, oxidation (steam auto-claving), surface passivation, electropolishing, and other modifications that serve to stabilize the surface of soft tissue coaptor 10. In exemplary embodiments, soft tissue coaptor 10 is coated with a bioactive agent and/or a medicant, for example, heparin, anti-inflammatories, anti-biotics, anti-bodies and/or steroids.

It will be understood by those in the art that the circular second configuration of soft tissue coaptor 10 shown in FIGS. 1A and 1B may alternatively comprise any number of configurations including, but not limited to ovalized, crescent and a custom patient specific configuration. The second configuration of soft tissue coaptor 10 may comprise a single loop or a plurality of loops (i.e., double overlap, triple overlap, etc.). In a similar manner, the second configuration of soft tissue coaptor 10 may comprise a partial loop (i.e., incomplete overlap) and be configured to clamp or pinch tissue rather than pierce tissue upon its deployment from the deployment device of the present invention. In yet another exemplary embodiment, the second configuration of soft tissue coaptor 10 may have a corkscrew configuration, for example, to eliminate the need for deploying multiple coaptors. A coaptor having a circular configuration may have an inner diameter of from about 0.1 mm to about 5 cm, or smaller or larger depending on the particular application, for example, 0.5 mm, 1.0 mm, and 2.0 mm. A coaptor having an ovalized configuration may provide additional features or functions such as being lower profile.

In addition, other cross sectional profiles in addition to the round nitinol wire may be used for the coaptor such as, but not limited to, a ribbon profile, polygonal profile, elliptical profile, square profile, triangular profile, oval profile or any other suitably shaped profile. An exemplary coaptor may have a cross sectional profile characterized by one or more grooves, bumps or ridges. A coaptor having a round wire type profile may have an diameter of from about 0.001 in to about 0.3 in, or smaller or larger depending on the particular application, for example, 0.002 in, 0.005 and 0.007 in diameters. A coaptor having a profile other than a round wire type profile may provide additional features or functions such as preventing the coaptor or clip from rotating and/or providing greater surface area to prevent ripping or migration of the coaptor clip. One exemplary embodiment of the coaptor may comprise a nitinol wire with a ribbon like profile that is set to have a circular shape or some other shape based on a patient's radiological image.

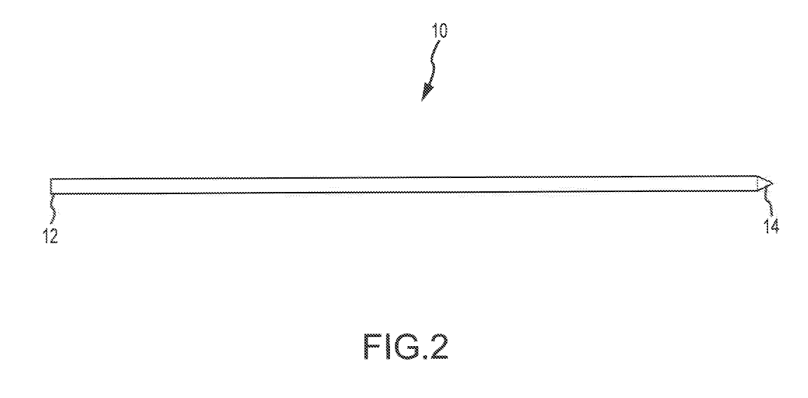

FIG. 2 shows a perspective view of the soft tissue coaptor of FIG. 1A shown in a deformed, straight line, shape which is the configuration it is in when loaded within the deployment device of the present invention. Shown in a straight line configuration, soft tissue coaptor 10 has a distal end 12 and a sharpened proximal end 14. It will be understood by those in the art that the straight line first configuration of soft tissue coaptor 10 may alternatively comprise any number of configurations. For example, as an alternative to being straight and rigid, the first configuration of coaptor 10 may comprise a bent or circular configuration before being implanted.

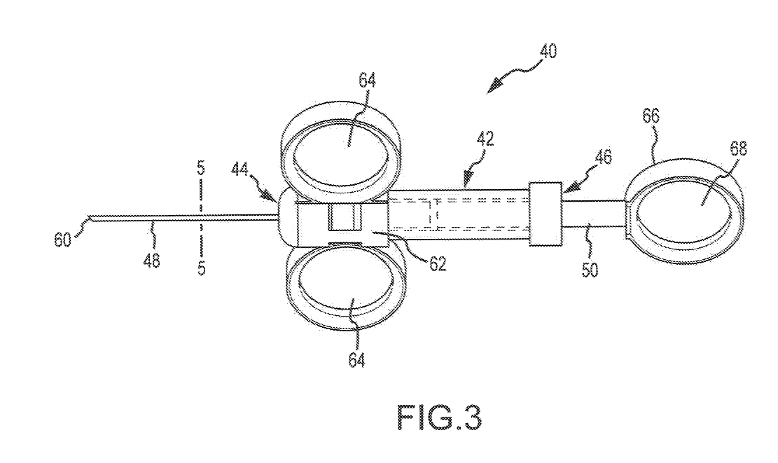

A perspective view of an exemplary deployment device 40 of the present invention is shown in FIG. 3. Deployment device 40 includes a hollow housing 42 having a first end 44 and a second end 46, a hollow deployment chamber or body 48 positioned within, and/or extending from, the first end 44 of the hollow housing 42, and an actuator mechanism 50 positioned within, and/or extending from, the second end 46 of the hollow housing 42. During use, one exemplary embodiment of the soft tissue coaptor 10 of the present invention is deformed into a straight line configuration and loaded into hollow deployment chamber 48. In a preferred embodiment, coaptor 10 is back-loaded into hollow deployment chamber 48 using a specifically fabricated device which straightens coaptor 10 as it is pushed into the lumen of hollow deployment chamber 48. The deformed straight line coaptor is contained within hollow deployment chamber 48 until it is deployed, or pushed from, the hollow deployment chamber 48 by the actuator mechanism 50.

After loading the shape memory material coaptor 10 into the hollow deployment chamber 48 in a straight configuration, the actuator mechanism 50 remains in an unemployed position. When a user manually actuates the deployment device 40, a mechanism for deploying coaptor 10 is engaged. In one exemplary embodiment, the deploying mechanism may include an ejector pin (not shown) that is guided down the hollow deployment chamber 48 and then pushes out the shape memory material coaptor 10. In this exemplary embodiment, the actuator mechanism 50 comprises an ejector pin (not shown) extending from an actuator rod (not shown) that is capable of fitting within the hollow deployment chamber 48 and pushing out the shape memory material coaptor 10.

In exemplary embodiments, the ejector pin is rigid, shape memory material, semisolid, or viscous. In yet other exemplary embodiments, pneumatic or hydraulic systems may be used to deploy the coaptor from the hollow deployment chamber.

By manually activating the actuator mechanism 50, a user can control the speed at which the coaptor deploys. Deployment of coaptor 10 can also be performed incrementally through the motions of the user. In the case of a biocompatible shape memory material such as nitinol, as coaptor 10 leaves the hollow deployment chamber 48, the coaptor 10 immediately starts to recover to its final circular shape. Deployment of coaptor 10 from the hollow deployment chamber 48 may occur with its proximal end resting on the surgical site or biting into and traveling through tissue surrounding the surgical site. It should be noted, however, that the final shape (i.e. second configuration) may not be circular in some embodiments but may instead comprise any number of configurations including ovalized and a custom patient specific configuration.

Once the coaptor 10 is fully deployed, the deployment device 40 can be removed leaving the coaptor 10 to hold a defect shut. While not required, the coaptor 10 can later be removed from the eye, if desired, by grabbing the coaptor 10 and pulling or by grabbing the coaptor 10 at two points with forceps, straightening the wire of the soft tissue coaptor or clip 10 by pulling the forceps away from one another, and then removing the straightened coaptor 10 from the eye. The elastic properties of the soft tissue coaptor 10 allow it to deform upon removal, thereby preventing tearing or damage to the surrounding tissue. The coaptor 10 is then removed from the same path through which it was deployed. Various other mechanical, adhesive and frictional anchoring mechanisms may be additionally or alternatively employed to assist in the removal of coaptor 10 post deployment. In exemplary embodiments, coaptor 10 comprises one or more of a hole, indentation, barb, eyelet or the like at its distal end to facilitate its removal.

It will be understood by those skilled in the art that hollow deployment chamber 48 may comprise any number of configurations or shapes for housing coaptors or clips having any number of different geometries. In exemplary embodiments, the inner cross-sectional dimension of hollow deployment chamber 48 conforms to the outer cross-sectional dimension of coapter 10. An advantage to conforming hollow deployment chamber 48 to coapter 10 is the ability to frictionally engage coapter 10 and prevent it from springing out prematurely during deployment. In accordance with yet another exemplary embodiment, the actuator mechanism, or any other component of the deployment device, can be rotated to rotate the coaptor during deployment of the coaptor. In an exemplary embodiment, hollow deployment chamber 48 is conformed to a coapter 10 having a profile other than a round wire type profile. In this manner, the system may be configured to provide additional features or functions such as preventing the coaptor or clip from rotating.

Hollow deployment chamber 48 may also have any number of different configurations or shapes for different suturing applications and/or varying procedures. For example, with a coaptor or clip having a ribbon like profile, hollow deployment chamber 48 may have ribbon shaped configuration to conform to the ribbon like profile of the coaptor or clip to house the coaptor/clip before deployment and to reduce rotation and entry profile upon deployment.

In addition to being straight and rigid with reference to FIGS. 3, 6 and 7A, hollow deployment chamber 48 could have bent, double bent configuration, or a curved configuration to a specified radius or dimension. In addition, hollow deployment chamber 48 may be semi-rigid so that it can be manipulated or bent by a user into any configuration, and in some embodiments, manipulated or bent during a procedure. Once bent or manipulated into a specific configuration, the hollow deployment chamber 48 would maintain that configuration at least until deployment of the coaptor/clip 10. After deployment of the coaptor/clip 10, hollow deployment chamber 48 could be remanipulated to any other configuration or shape.

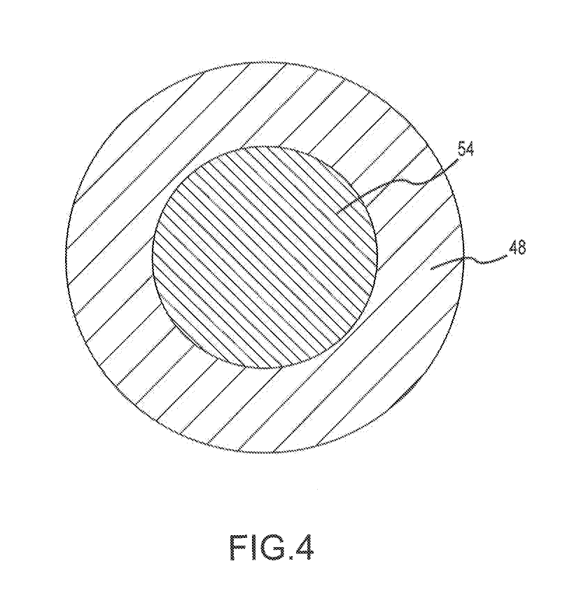

A cross-sectional view of an exemplary deployment device 40 taken along line 5-5 of FIG. 3 is shown in FIG. 4. As previously described above, actuator mechanism 50 may include a mechanism for deploying the coaptor 10 of the present invention from the hollow deployment chamber 48 once its proximal end 60 is in a desired position. With reference to FIG. 4, the deploying mechanism may comprise an ejector pin 54 which fits within the hollow deployment chamber 48. Ejector pin 54 preferably comprises a long thin column made from a rigid material that can resist buckling from the resistive force applied to actuator mechanism 50, e.g., directly or indirectly via an actuator rod, when deploying the coaptor 10 from the hollow deployment chamber 48, but could be semisolid, viscous, pneumatic, or hydraulic in nature.

Actuator mechanism 50 may also include a mechanism for anchoring the distal end 12 of the coaptor 10 of the present invention within the hollow deployment chamber 48 such that it can be secured, drawn back or retracted within the hollow deployment chamber 48 until it is ready to be deployed or pushed through a distal end of hollow deployment chamber 48 when it is deployed. This action may be accomplished by a clasp (not shown) on the distal end of the coaptor 10. The clasp may be engaged or disengaged by a lever (not shown) on hollow housing 42. Various other mechanical, adhesive and frictional anchoring mechanisms may be additionally or alternatively employed to secure, draw back or retract coaptor 10 within the hollow deployment chamber 48 until it is ready to be deployed.

Actuator mechanism 50 may further include an ejector pin 54 contained within, and/or extending from an actuator rod (not shown), wherein the ejector pin 54 is capable of fitting within the hollow deployment chamber. In exemplary embodiments, the proximal end of ejector pin 54 may be completely contained within the hollow deployment chamber 48 at all times and the hollow deployment chamber may include a sharpened proximal end 60 for piercing tissue to allow access to a deployment site.

In exemplary embodiments, hollow deployment chamber 48 comprises a needle. In exemplary embodiments, hollow deployment chamber 48 comprises a beveled needle. An exemplary needle may comprise a 25-gauge hypodermic needle having an internal diameter of 0.20 mm (0.01025 inch) to receive coaptor wire diameters up to 0.007 inch with some tolerance for movement. Yet another exemplary needle may comprise a 30-gauge hypodermic needle having an internal diameter of 0.159 mm (0.00625 in) to allow passage of coaptor wire diameters up to 0.002 inch. Furthermore, any suitable size needle and coaptor diameters may be used.

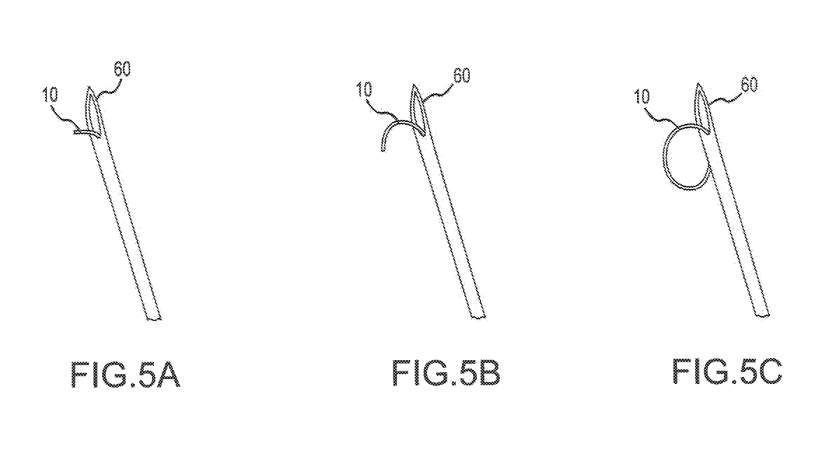

With reference to FIGS. 5A-5C, in preferred embodiments, coaptor 10 deploys in a curved direction at the base of and away from the bevel of sharpened proximal end 60. In this manner, a user can control the direction coaptor 10 is deployed. By way of non-limiting example, the actuator mechanism, or any other component of the deployment device, can be rotated to control the direction coaptor 10 is deployed.

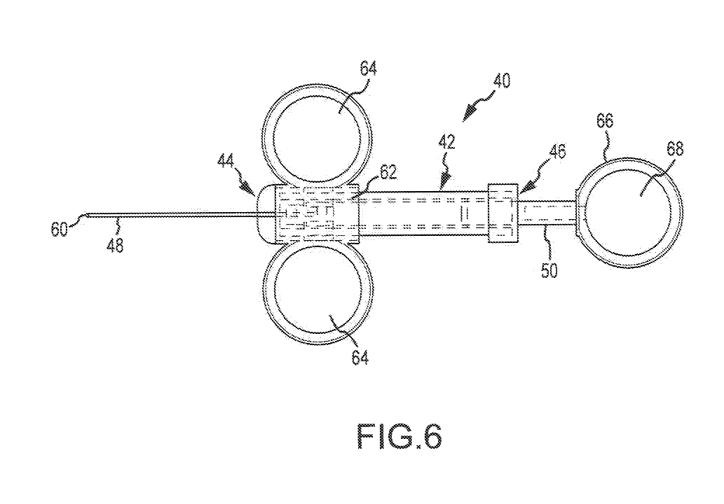

A top plan view of an exemplary deployment device 40 of the present invention is shown in FIG. 6. Deployment device 40 may further comprise a finger support 62. In exemplary embodiments, finger support 62 is attached to an outer surface of hollow housing 42 for supporting a user's fingers while employing the actuator mechanism 50 of deployment device 40 to allow greater control while utilizing the deployment device 40. Finger support 62 may include two openings 64 on opposite sides of the hollow housing 42 for inserting a user's fingers therethrough. Finger support 62 helps promote greater stability during deployment of the biocompatible soft tissue coaptor 10 of the present invention from the deployment device 40 of the present invention. In addition, actuator mechanism 50 may include a thumb support 66 attached to the actuator mechanism 50 for supporting a user's thumb while employing the actuator rod 56 of the actuator mechanism 50. Thumb support 66 may comprise a post or include an opening 68 for inserting a user's thumb therethrough.

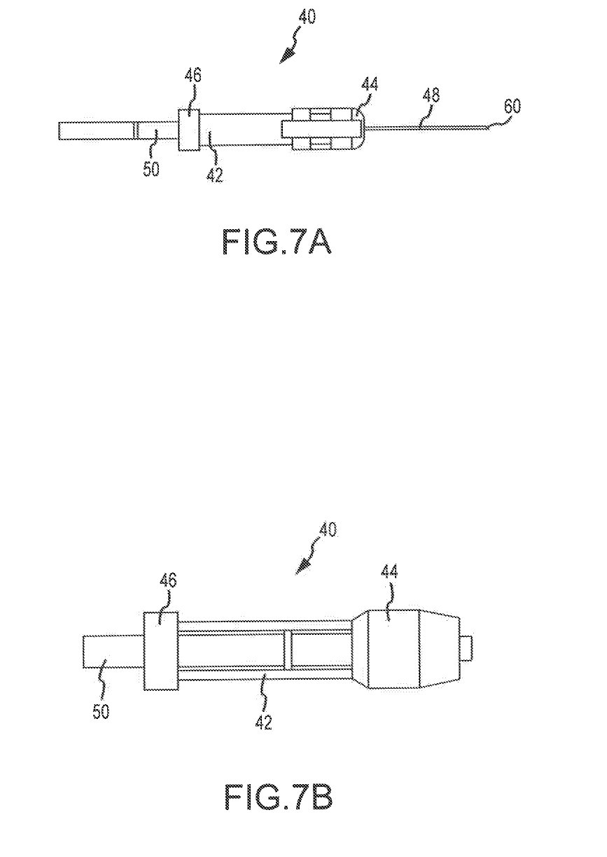

Views of additional exemplary deployment devices 40 of the present invention are shown in FIGS. 7A and 7B.

The deployment device 40 of the present invention may be constructed such that it can receive, retain, and deliver multiple coaptors 10. Multiple coaptors 10 would be loaded into the deployment device 40 in series or in parallel allowing a user to deploy the coaptors 10 one at a time without requiring reloading of the deployment device 40, or switching devices, thereby eliminating any interruption in the procedure. In another embodiment, the deployment device 40 of the present invention may be constructed such that material for coaptors 10 can be controllable advanced and cut to a desired length as it is delivered by the deployment device 40.

In various embodiments, the deployment device 40 of the present invention may be configured to be reusable and/or have removable hollow deployment chambers 48. By way of illustration, the deployment device 40 may be constructed such that it can receive and retain multiple hollow deployment chambers 48, for example, by incorporating a luer-lock type system, such as shown at first end 44 of hollow housing 42 of deployment device 40 of FIG. 7B.

Exemplary embodiments of the present invention were engineered and tested in simulated surgical settings by the inventors and exhibited surprising improvements, e.g., in terms of surgical times and opening pressures. In various micro-surgical scenarios, an exemplary injectable system proved to be five to twenty times more efficient and wound strengths over three times that of conventional suturing. Further, exemplary shape-memory alloy clips could be forced to failure and then recover repeatedly, unlike conventional sutures. An exemplary surgical tool for injecting shape-memory alloy clips in a microsurgical setting proved to be quicker, stronger, and technically easier than conventional suturing.

### In Vitro Testing

The various materials tested as potential candidates for the clip were nylon, enamel coated copper, and nitinol. A standard filament of 1.5 inch length filament of each material was grasped with a needle driver and the distal end inserted into the iris of an enucleated porcine eye. In this scenario, only the nitinol filaments remained rigid and did not buckle upon insertion into tissue.

TABLE 1
Ex-vivo testing of candidate materials for clip. Various diameter wires
of nylon, enamel coated copper, and nitinol were tested for their ability
to pass through tissue without buckling. Of all the materials and sizes
tested, only nitinol was able to maintain its rigidity without buckling.
Clip Material Rigidity Testing

| Material | Filament Diameter (inches) | Result |
|---|---|---|
| Nylon | 0.005 | Buckling |
| Nylon | 0.010 | Buckling |
| Enamel coated copper | 0.005 | Buckling |
| Enamel coated copper | 0.010 | Buckling |
| Nitinol | 0.005 | No buckling |
| Nitinol | 0.010 | No buckling |

Using an electromechanical testing setup, 3 gauges of nitinol wire (0.002 inch, 0.005 inch, 0.007 inch) and two types of 10-0 suture (nylon and prolene) were tested for tensile properties, recording strain to failure and break force for each.

TABLE 2
Electromechanical tensile testing. Comparison of strain to failure
and break force for three different gauges of nitinol wire and
two types of suture commonly used in ophthalmic surgery.
Tensile Properties of Nitinol Wire and Suture

| | Average Strain to Failure | Average Break Force (N) |
|---|---|---|
| 0.002 inch Nitinol | 50.7 | 2.635 |
| 0.005 inch Nitinol | 57.0 | 17.427 |
| 0.007 inch Nitinol | 59.9 | 32.787 |
| 10-0 Nylon | 90.5 | 0.461 |
| 10-0 Prolene | 123.8 | 0.541 |

For the pupilloplasty and IOL fixation procedures described below, 10-0 prolene is the standard suture typically used, compared to the 0.002 inch nitinol clip that was employed for comparison. The nitinol has a break force of 2.635 N compared to 0.541 N for the prolene suture, a five fold difference. In external suturing, such as corneal wound closure, 10-0 nylon is the standard suture typically used, compared to 0.005 inch nitinol clip. In this instance, the nitinol had an average break force of 17.5 N compared to 0.46 N for the suture, a 38-fold difference. Further, the suture material has a tendency to lose its shape and elongate in a linear fashion prior to breakage. In contrast, all gauges of nitinol demonstrated a resistance to shape deformation under strain.

### Ex-vivo Corneal Wound Strength Testing

The ability of the exemplary coaptor to close a corneal wound and maintain globe integrity without leakage was tested and compared to a conventional suture. For this portion of the testing, six enucleated porcine eyes were used and a 3.2 mm corneal wound was created in each eye using a beveled surgical blade. Three of the eyes were sutured closed with 10-0 nylon suture, and three of the eyes were closed with the exemplary coaptor system, using a 0.005 inch diameter nitinol wire, double overlap clip and a 27-gauge delivery needle. Pressure testing was then done by using a 50 cc syringe filled with saline solution which was injected into the anterior chamber of each eye via a 27-gauge butterfly needle. Pressure recordings and concurrent video recordings were taken using the ADI system 8/30 powerlab and MLT844 physiological pressure transducer system. Each globe was then subjected to a graded intraocular pressure increase, and each wound monitored for leakage by applying orange dye (fluoroscein) to the external surface of the wound. Recordings were taken at the time of initial wound leak, and then the pressures escalated further and recordings made of suture breakage and wound failure.

Using the pressure measurement system described above, the exemplary coaptor was tested against a conventional suture for wound strength in six enucleated pig eyes. For each eye, a 3.2 mm corneal wound was created, and found to leak without any surgical wound closure at a mean intraocular pressure of 15 mm Hg. The corneal wound in three eyes was dosed with a single, 10-0 nylon suture using a standard surgeon's knot. The sutures were tied by an experienced ophthalmic surgeon in 63 seconds for the first eye, 58 seconds for the second eye, and 59 seconds for the third. The first eye leaked at a pressure of 20 mm Hg with the suture remaining intact. Attempts to raise the pressure higher were unsuccessful because of the rapid leakage from the wound. The second conventional suture eye was able to maintain a pressure of 68 mm Hg, and the third a pressure of 50 mm Hg, before the sutures broke and the wounds failed.

The fourth eye, closed with the exemplary coaptor in a time of 20 seconds, was able to maintain a pressure of 157 mm Hg without leakage or failure of the clip. A small leak around the infusion needle prevented higher pressures to be tested in this eye. The fifth eye, closed with the exemplary coaptor in a time of 21 seconds, was able to maintain a pressure of 174 mm Hg, at which point the clip opened and the wound leaked. Notably, once the pressure fell below 57 mm Hg, the shape memory clip returned to its original configuration and closed the wound. This eye was then retested, and the clip was able to hold the wound again to pressures of 155 mm Hg. The sixth eye was closed in a time of 21 seconds, and the wound able to maintain 150 mm Hg.

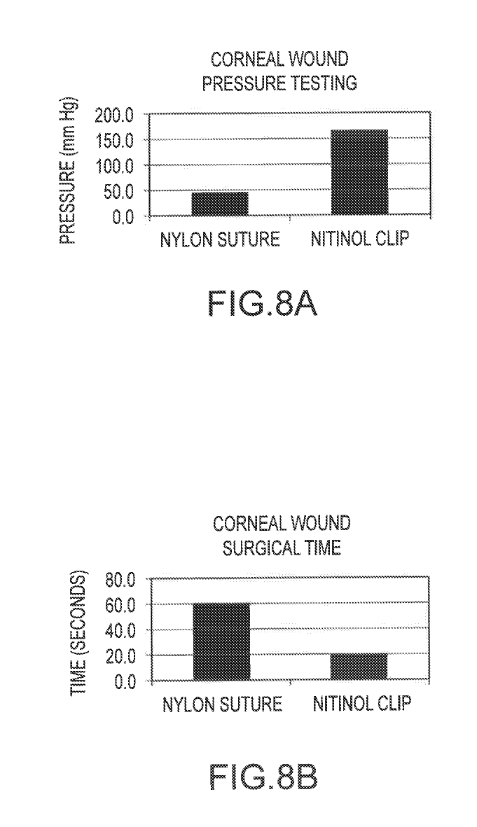

On average, the surgical time to close the wounds was 60 seconds in the suture group and 21 seconds in the exemplary coaptor group, which was statistically significant (t-test, p=0.001); Further, the opening pressure of the wounds averaged 46 mm Hg in the suture group compared to 160 mm Hg in the alloy clip group, which was also statistically significant (t-test, p=0.005). In aggregate, the exemplary coaptor demonstrated an ability to withstand pressures approximately 3.5 times that of conventional suture, with the added ability to fail and then recover and reestablish wound integrity—in contrast to suture which breaks and wound integrity is not recoverable. Further, the exemplary coaptor was about three times faster to deploy into the corneal wound than conventional suturing techniques. The data is depicted graphically in FIGS. 8A and 8B.

A 2 mm iris defect was created in an enucleated porcine eye using Vanass scissors. Two limbal paracenteses were made for instrument entry into the anterior chamber and a forceps used to stabilize the mid-peripheral iris. The 30-gauge injector was employed to puncture both iris leaflets, and the shape memory alloy clip deployed to fixate the leaflets together. The iris was then subjected to a mechanical stress test as well as an intraocular pressure test, using the same method as described above. The globe was then dissected, the fixation of the iris confirmed from a posterior view, and second mechanical stress test performed.

Using the surgical set-up described above, pupilloplasty was performed in two enucleated porcine eyes. One eye was repaired with conventional anterior segment techniques and 10-0 prolene suture on a CIF-4 needle. In this eye, three 1 mm cortical incisions were made and a modified Seipser knot was utilized. The total surgical time was 19 minutes 38 seconds for a single suture. A mechanical stress test was performed by pulling on the iris leaflets with intraocular forceps. There was no slippage of the wound or the suture knot.

The pupilloplasty in the second eye was repaired using a 0.005 inch nitinol wire diameter exemplary coaptor wire, heat molded into a circular conformation with a diameter of 0.5 mm, double overlap, and delivered with a 30-gauge injector system. Two corneal incisions were made, namely a first incision for the injector and a second incision for intraocular forceps to stabilize the iris tissue. The needle tip of the injector system was passed through both leaflets of the iris and then the clip was deployed. The total surgical time was 1 minute 18 seconds. A mechanical stress test was performed as described above, and there was no slippage of the iris wound or the nitinol clip.

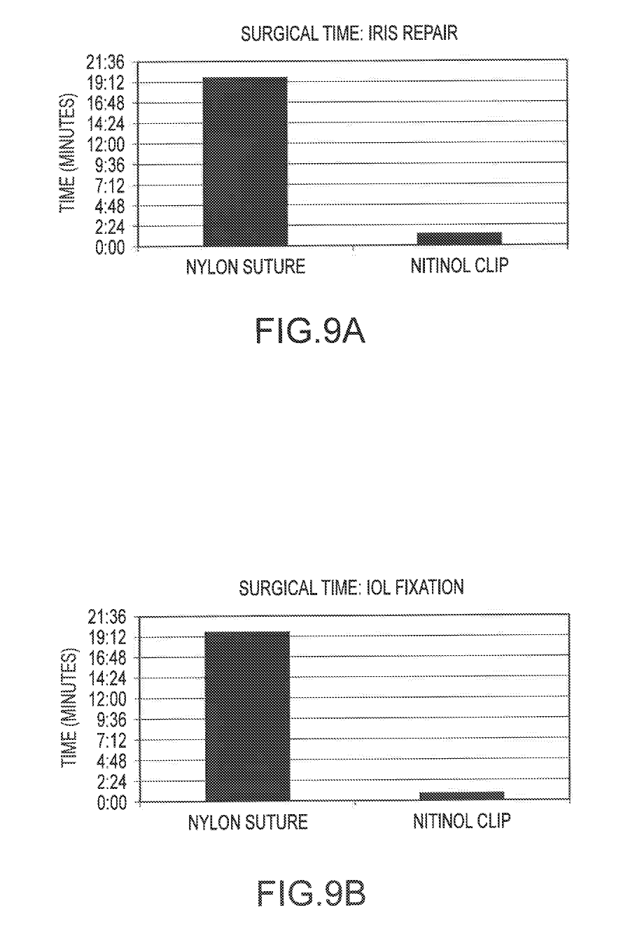

In short, the exemplary coaptor delivery system was nearly 15 times faster to deploy than conventional suturing techniques in the setting of iris repair in the tight surgical confines of the anterior segment, with no qualitative difference in wound strength on mechanical stress testing. Further, one less corneal wound was required for the exemplary coaptor technique to allow for surgical instruments and maneuvering, meaning a less invasive surgical approach. The data is represented graphically in FIG. 9A.

### Ex-vivo Intraocular Lens Fixation

An intraocular lens was inserted into an enucleated porcine eye. Two limbal paracenteses were made and a forceps used to stabilize the IOL haptic beneath the mid-peripheral iris. The 30-gauge injector was employed to puncture the iris adjacent to the haptic, and the shape memory alloy clip deployed to fixate the IOL to the iris. The lens was then subjected to a mechanical stress test. The globe was then dissected, the fixation of the haptic confirmed from a posterior view, and second mechanical stress test performed.

Using the surgical set-up described above, a single enucleated pig eye into which a standard acrylic intraocular lens (Model NM60D3, Alcon, Fort Worth, Tex.) had been inserted into the cilliary sulcus was used. The surgical procedure to fixate the lens, not including initial placement of the intraocular lens, was completed in 60 seconds. The shape memory clip was visible anteriorly, directly over the haptic of the IOL. During mechanical stress testing, the IOL was found to be stable and no slippage of the haptic or clip observed. After a Miyake-Apple preparation of the anterior segment, the haptic was seen to be firmly attached to the iris by the shape memory alloy clip. A second mechanical stress test from this posterior view of the surgical site demonstrated a secure fixation of the haptic.

The surgical time for this procedure was compared to a modified Seipser knot, as this is a conventional method of intraocular lens fixation. In brief, the exemplary coaptor delivery system was nearly 20 times faster than conventional suturing in the setting of a closed anterior chamber of the eye for intraocular lens fixation surgery. The data is represented graphically in FIG. 9B.

### Biocompatibility Testing

Surgical testing in vivo was done to test exemplary embodiments of the device. Six eyes of three pigs underwent anterior segment ophthalmic to compare the shape memory alloy clip and conventional suture. All eyes were prepped and draped in the usual surgical manner, and two standard 1 mm paracenteses were created with a side-port blade. Three eyes received the shape memory clip and three eyes received a standard 10-0 prolene suture, all of which were placed in the iris. Prior to the surgeries and prior to enucleation, electroretinograms were performed on all six eyes. Post-operatively, there was no difference between the eyes in terms of inflammation, no infections occurred, and there was no incidence of cataracts. After two months, the eyes were encleated and examined with specular microscopy, anterior segment optical coherence tomography, and histologically. There was no statistical difference between the two groups in terms of corneal thickness, endothelial cell count, specular microscopy, or electroretinography.

The described embodiments are to be considered in all respects only as illustrative of the current best modes of the invention known to the inventors at the time of filing this application, and not as restrictive. Although the several embodiments shown here include specific components and features, these are provided in order to show examples of the present embodiments of the invention. The scope of this invention is, therefore, indicated by the appended claims rather than by the foregoing description. All devices and processes that come within the meaning and range of equivalency of the claims are to be embraced as being within the scope of the patent.

1. A deployment device for delivering and securing a coaptor comprising:
a cylindrical hollow housing having a first end and a second end;
a cylindrical hollow deployment chamber positioned within, and extending from, the first end of the hollow housing;
an actuator mechanism positioned within, and extending from, the second end of the hollow housing; and
at least one biocompatible coaptor contained within the hollow deployment chamber wherein said at least one biocompatible coaptor comprises shape memory material.

2-4. (canceled)

5. The deployment device of claim 1 wherein the shape memory material biocompatible coaptor comprises a first configuration when loaded and contained within the hollow deployment chamber and a custom configuration when it is completely deployed from the hollow deployment chamber.

6. The deployment device of claim 5 wherein the first configuration comprises a straight line configuration, and the custom configuration comprises a circular configuration.

7. The deployment device of claim 1 wherein the shape memory material biocompatible coaptor comprises a distal end and a sharpened proximal end for piercing tissue upon its deployment.

8. The deployment device of claim 1 further comprising multiple coaptors contained within the deployment device such that more than one coaptor can be deployed during a single procedure without reloading the deployment device.

9. The deployment device of claim 1 wherein the actuator mechanism comprises a mechanism for anchoring a distal end of a coaptor within the deployment device and a mechanism for deploying the coaptor from the deployment device once the deployment device is in a desired position.

10. The deployment device of claim 9 wherein the actuator mechanism can be rotated to rotate the coaptor during deployment of the coaptor.

11. The deployment device of claim 9 wherein the mechanism for deploying the coaptor comprises an ejector pin which fits within the hollow deployment chamber.

12. The deployment device of claim 11 wherein the ejector pin comprises a long thin column made from a rigid material that can resist buckling from the resistive force applied to the actuator mechanism when deploying the coaptor from the hollow deployment chamber.

13. The deployment device of claim 11 wherein the actuator mechanism further comprises an actuator rod and the ejector pin extending from the actuator rod.

14. The deployment device of claim 11 wherein the proximal end of the ejector pin is completely contained within the hollow deployment chamber at all times.

15. The deployment device of claim 1 wherein the hollow deployment chamber comprises a sharpened proximal end for piercing tissue to allow access to a deployment site.

16. The deployment device of claim 1 further comprising a finger support attached to an outer surface of the hollow housing for supporting a user's fingers while employing the actuator mechanism of the deployment device to allow greater control while utilizing the deployment device.

17. The deployment device of claim 16 wherein the finger support comprises two openings on opposite sides of the hollow housing for inserting a user's fingers therethrough.

18. The deployment device of claim 1 further comprising a thumb support attached to the actuator mechanism for supporting a user's thumb while employing the actuator mechanism.

19. The deployment device of claim 18 wherein the thumb support comprises an opening for inserting a user's thumb therethrough.

20. The deployment device of claim 1 wherein the actuator mechanism comprises a geared down lever system such that the actuator is articulated through compression of the lever.

21. The deployment device of claim 1 further comprising a mechanism for integration with a robotic arm in order to perform remote surgery.

22. A coaptor comprised of a shape memory material having properties that allow it to be deformed into a first straight line configuration for loading into a deployment device and a circular configuration upon deployment from the deployment device.

23. The soft tissue coaptor of claim 19 wherein the soft tissue coaptor comprises a distal end and a sharpened end for piercing tissue upon its deployment.

24-25. (canceled)
